(19) United States
(12) Patent Application Publication
(10) Pub. No.: US 2002/0061840 A1
(43) Pub. Date: May 23, 2002

(54) Tripeptide compounds useful as selective inhibitors of aminopeptidase A and corresponding pharmaceutical compositions

(76) Inventors: Bernard Roques, Paris (FR); Marie-Claude Fournie-Zaluski, Paris (FR); Laurent Bischoff, Paris (FR); Christelle David, Nanterre (FR); Catherine LLorens-Cortes, Bures sur yvette (FR)

Correspondence Address: LARSON & TAYLOR, PLC, 1199 NORTH FAIRFAX STREET, SUITE 900, ALEXANDRIA, VA, 22314, US

(21) Appl. No.: 09/908,977

(22) Filed: Jul. 20, 2001

Related U.S. Application Data
(63) Continuation-in-part of application No. PCT/FR00/00112, filed on Jan. 19, 2000.

(51) Int. Cl. A61K 38/06; C07K 5/08

(52) U.S. Cl. 514/7; 514/18; 530/331

(57) ABSTRACT

The invention concerns a compound of general formula (I) wherein: R₁ represents an alkyl, alkenyl or alkynyl chain, or a cycloalkyl, or (cycloalkyl)alkyl group substituted by at least a COOH, SO₃H, PO₃H₂ or tetrazolyl group; R₂ represents an alkyl chain, or an aryl, arylalkyl, cycloalkyl, (cycloalkyl)alkyl, (heteroaryl)alkyl group substituted or not by at least a OH, OR, SR′, NH₂, NHR′, guanidinyl, COOH, CONH₂ group, or a halogen atom; R₃ represents a hydrogen atom or a methyl group; R₄ represents a) an alkyl chain, an aryl, alrylalkyl, cycloalkyl, (cycloalkyl)alkyl, (heteroalkyl)alkyl, heterocycloalkyl or (heterocycloalkyl)alkyl group substituted by at least a CONH₂, SO₃H, SO₂NH₂, PO₃H₂ or tetrazolyl group, (b) C₂-C₆ alkyl chain, an aryl, arylakyl, cycloalkyl, (cycloalkyl)alkyl, (heteroaryl)alkyl, heterocycloalkyl, (heterocycloalkyl)alkyl group substituted by at least a CO₂H group capable of being protected as described above; or c) R₃ and R₄ can together form a heterocyclic compound, with 5 to 6 links, substituted by at least a CO₂H, CONH₂, SO₃H, SO₂NH₂ or PO₃H₂ group; X represents a CONH or CH₂NH; and Z represents a OH, OCH₂-C₆H₅ or NR″R′″ group.

The present invention relates to new compounds of therapeutic interest which may be used particularly as selective inhibitors of aminopeptidase A and the corresponding pharmaceutical compositions.

Aminopeptidase A is a zinc ectopeptidase which is very specifically involved in the degradation of peptide substrates having an Asp or Glu residue in the N-terminal position.

Of the biologically active peptides of the central or peripheral nervous system, the C-terminal octapeptide of cholecystokinine CCK₈ and angiotensin II are precisely physiological substrates of aminopeptidase A.

CCK₈ interacts with two types of receptors, the CCK-A sites located chiefly at the periphery and the CCK-B receptors preferably located in the central nervous system. At the peripheral level, CCK₈ stimulates the contractions of the bile duct and ileum, increases intestinal and gastric motility, promotes the secretion of pancreatic enzymes. In the central nervous system, CCK₈ regulates alimentary behaviour, promotes the release of hypophyseal hormones and leads to behavioural changes linked with anxiety.

Consequently, the use of CCK₈ degradation inhibitors constitutes an effective method of intervening in these different physiological processes and thereby changing them.

As for angiotensin II and its metabolite, angiotensin II, they are part of the angiotensinergic cascade, interact with the receptors ATI and ATII and intervene in a different way in the central and peripheral control of blood pressure and the release of hypophyseal hormones such as vasopressin.

Consequently, in this particular case, it is possible to modulate arterial pressure and the release of vasopressin by inhibiting the degradation of angiotensin II into angiotensin III. This would be a therapeutic method of treating essential and secondary arterial hypertension, cardiac and renal failure, disorders of hydrodynamic homeostasis, myocardial infarction and proteinuria in diabetics.

APA inhibitors have already been proposed in the literature. They correspond more particularly to the following general formula A:

H₂N—CH(R_{A})CH₂SH A

wherein R_{A} preferably denotes an aliphatic chain substituted by a negatively charged group (Wilk and al. (1990) Neuropeptides 16, 163-168; Chauvel and al. (1994) J. Med. Chem. 37,1339-1346, J. Med. Chem. 37, 2950-2956).

However this family of compounds has the major disadvantage of having no selectivity with regard to aminopeptidase A.

Parallel to the inhibition of aminopeptidase A, these compounds also inhibit aminopeptidase N. Thus their Ki are, at best, of the order of 10⁻⁷ M on APA and their selectivity factor with regard to APN is relatively low (less than 100).

It is clear that this non-selective behaviour constitutes a handicap from the therapeutic point of view.

The present invention sets out precisely to propose a new family of compounds which advantageously has a selective nature with regard to APA.

More precisely, the present invention relates to a compound of general formula I:

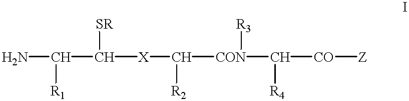

wherein:

R₁ denotes an alkyl, alkenyl or alkynyl chain, or a cycloalkyl, or (cycloalkyl)alkyl group substituted by at least one

—COOH group, optionally esterified by an alkyl group comprising 2 to 12 carbon atoms,

SO₃H group, optionally protected by a pentyl group,

PO₃H₂ group, optionally substituted by a (—CH₂CH₂SCOR₅) group, with R₅ representing a C₁-C₄ alkyl group, a phenyl or benzyl group, or

tetrazolyl group.

R₂ denotes an alkyl chain, or an aryl, arylalkyl, cycloalkyl, (cycloalkyl)alkyl, (heteroaryl)alkyl group which may or may not be substituted by at least one OH, OR, SR′, NH₂, NHR′, guanidinyl, COOH or CONH₂ group, or a halogen atom selected from among F, Cl, Br or I with R′ representing a straight-chain or branched C₁₋₄ alkyl group.

R₃ denotes a hydrogen atom or a methyl group,

R₄ denotes

an alkyl chain, an aryl, arylalkyl, cycloalkyl, (cycloalkyl)alkyl, (heteroaryl)alkyl, heterocycloalkyl or (heterocycloalkyl)alkyl group substituted by at least one CONH₂, SO₃H, SO₂NH₂, PO₃H₂ or tetrazolyl group, with the groups SO₃H and PO₃H₂ optionally protected as described above,

a C₂₋₆ alkyl chain, an aryl, arylalkyl, cycloalkyl, (cycloalkyl)alkyl, (heteroaryl)alkyl, heterocycloalkyl or (heterocycloalkyl)alkyl group substituted by at least one CO₂H group optionally protected as described above, or

R₃ and R₄ may together constitute a 5- or 6-membered heterocyclic compound, substituted by at least one CO₂H, CONH₂, SO₃H, SO₂NH₂ or PO₃H₂ group with the groups CO₂H, SO₃H and PO₃H₂ optionally protected as described above,

X denotes a group CONH or CH₂NH,

Z denotes a group OH, OCH₂-C₆H₅ or NR″R′″ wherein R″ and R′″ may denote independently of one another a hydrogen atom or an alkyl, aryl or arylalkyl group, where R″ and R′″ may constitute, together with the nitrogen atom, a 5- or 6-membered heterocycle possibly having a second heteroatom selected from among O, S and N,

R denotes a hydrogen atom or a group of formula II

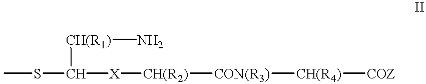

corresponding to the symmetric disulphide of the inhibitor wherein R₁, R₂, R₃, R₄, X and Z are defined as hereinbefore, and the derivatives thereof.

The definitions of the various groups proposed in general formula I of the compounds claimed are given in the list which follows. These definitions apply to the terms as used throughout this text (unless they are restricted to precise examples) both individually and as part of a wider group.

Thus, within the scope of the invention, unless stated to the contrary:

Alkyl denotes a straight-chain or branched saturated aliphatic chain having I to 6 carbons.

Alkenyl denotes a straight-chain or branched aliphatic chain with 2 to 6 carbons having a double bond.

Alkynyl denotes a C₂₋₆ aliphatic chain having a triple bond.

Cycloalkyl denotes a saturated C₃₋₆ hydrocarbon ring.

Aryl denotes an aromatic C₆ ring which may or may not be fused and/or may or may not be substituted by on or two other aromatic C₆ rings.

Heteroaryl denotes an aromatic 5- or 6-membered heterocycle containing 1 or 2 heteroatoms selected from among N, S, O, optionally fused or substituted by an aromatic C₆ ring.

Heterocycloalkyl denotes a saturated 5- or 6-membered heterocycle containing 1 or 2 heteroatoms selected from N, S, O optionally fused or substituted by an aromatic C₆ ring.

For the purposes of the present invention, the term “derivatives” refers particularly to the addition salts of the compounds of general formula (I) obtained with pharmacologically acceptable organic or inorganic acids. These may be for example salts such as the hydrochloride, hydrobromide, sulphate, nitrate, borate, phosphate, methane sulphonate, acetate, fumarate, succinate, ascorbate, oxalate, lactate, pyruvate, citrate, tartrate, maleate, malonate, benzoate, diaminobenzene sulphonate, cromoglycate, benzene sulphonate, cyclohexane sulphonate, toluene sulphonate, dipropyl acetate, glucose-1 phosphate, palmoate and palmitate.

Of these derivatives, mention may also be made of the dimers of compounds of general formula 1, consisting of two molecules of compounds of general formula 1, identical or different, coupled to each other at their respective sulphur atoms. In this particular case, R denotes the group of formula II identified hereinbefore.

Similarly, the present invention also includes the different enantiomeric forms of the compounds claimed.

In fact, the compounds of formula (I) which have a number of asymmetric carbons are present in the form of either racemic or diastereomeric mixtures or in the form of the pure stereoisomers.

The optically pure compounds may be isolated by enantioselective syntheses or resolution by chiral amines. In the case of methods of preparation which produce mixtures of stereoisomers, separation by semi-preparative column HPLC (Kromasil C₁₈, 20×250 mm, CH₃CN—H₂O ) is carried out, allowing each stereoisomer to undergo separate biochemical and pharmacological investigation.

Of these stereoisomers, those having an absolute (S) or (R) configuration on the carbon carrying the group R₁, an (S) or (R) configuration on the carbon carrying the —SR function, and an (S) configuration on the carbons carrying the groups R₂ and R₄ are preferred.

According to a preferred embodiment of the invention, the compounds claimed correspond to general formula II wherein

R₄ denotes an alkyl chain, an aryl, arylalkyl, cycloalkyl, (cycloalkyl)alkyl, (heteroaryl)alkyl, heterocycloalkyl or (heterocycloalkyl)alkyl group substituted by at least one CONH₂, SO₃H, SO₂NH₂, PO₃H₂ or tetrazolyl group, with the groups SO₃H and PO₃H₂ optionally being protected, or

R₄ constitutes with R₃ a 5- or 6-membered heterocyclic compound, substituted by at least one CO₂H, CONH₂, SO₃H, SO₂NH₂ or PO₃H₂ group with the groups CO₂H, SO₃H and PO₃H₂ optionally protected as described above.

Of particular interest are the compounds claimed wherein R₄ and R₃ together constitute a 5- or 6-membered heterocyclic compound substituted by at least one CO₂H, CONH₂, SO₃H, SO₂NH₂ or PO₃H₂ group with the groups CO₂H, SO₃H and PO₃H₂ optionally being protected as described above.

According to a preferred embodiment of the invention, the compounds claimed correspond to general formula II wherein X denotes a CONH function and more preferably R denotes a hydrogen atom.

More preferably, R₂ denotes an alkyl or arylalkyl chain which is optionally substituted, preferably by a hydroxyl group.

The following may be mentioned as examples of compounds according to the present invention:

N-[[(2S,3R)- and (2R,3R)-3-amino-2-mercapto-5-sulphonate]pentanoyl]-L.Tyr-L.Sal-OH

N-[[(2S,3R)- and (2R,3R)-3-amino-2-mercapto-5-sulphonate]pentanoyl]-L.Tyr-L.hSal-OH;

N-[[(2S,3R)- and (2R,3R)-3-amino-5-carboxy-2-mercapto]pentanoyl]-L.IIe-L.(3R)(3-COOH)Pro-OH;

N-[[(2S,3R)- and (2R,3R)-3-amino-5-phosphonate-2-mercapto]pentanoyl]-L.IIe-L.Glu-OH;

the N-[[2S,3R) and (2R,3R), 3-amino-2-mercapto-5-sulphonate]pentanoyl]-L.IIe-L.Sal-OH;

N-[[(2S,3R)- and (2R,3R)-3-amino-2-mercapto-5-sulphonate]pentanoyl]-L.IIe-L.(3R)(3—COOH)Pro-OH;

N-[[(2S,3R)- and (2R,3R)-3-amino-2-mercapto-5-sulphonate]pentanoyl]-L.IIe-L.(3S)(3-COOH)Pro-OH; and

N-[[(2S,3R)- and (2R,3R)-3-amino-2-mercapto-5-sulphonate]pentanoyl]-L.IIe-L.Glu-NH₂.

In the above compounds, the abbreviations Sal and hSal denote the sulphoalanine and homosulphoalanine groups, respectively.

The compounds claimed may be obtained by various methods of synthesis depending on the definition of the group X.

The present invention also relates to a process which may be used to prepare the compounds of general formula (I) wherein X denotes a CONH group comprising at least coupling an ester dipeptide of general formula III

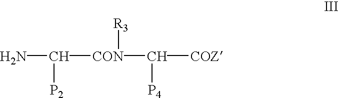

wherein

P₂ and P₄ correspond to protected forms of R₂ and R₄,

R₃ is as hereinbefore defined and

Z′ denotes a group OC(CH₃)₃, OCH₂-C₆H₅ or NR″R′″ wherein R″ and R′″ independently of one another may denote a hydrogen atom or an alkyl, aryl or arylalkyl group, while R″ and R′″ may constitute, together with the nitrogen atom, a 5- or 6-membered heterocycle possibly having a second heteroatom selected from among O, S and N,

with a compound of general formula IV

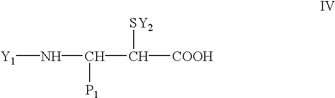

wherein:

Y₁ denotes a protecting group

Y₂ denotes a protecting group and

P₁ denotes a protected form of R₁, under conditions suitable to produce compound V

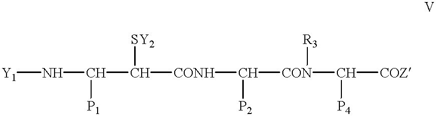

and, by deprotecting it, obtain said compound of general formula 1.

The coupling reaction is carried out in conventional manner in an organic solvent, in the presence of a coupling agent and a tertiary amine, at a temperature of the order of 20° C.

Generally, the coupling of the acid of formula (IV) with the amine of general formula (III) is carried out by working in an organic solvent such as dichloromethane or dimethylformamide, using as coupling reagent a BOP-type compound [benzotriazol-1-yl-oxy-tris(dimethylamino)-phosphonium -hexafluorophosphate], HATU [O-(7-azabenzotriazol-1-yl)1,1 ,3,3-tetramethyluronium hexafluorophosphate], or TFFH [tetramethylfluoro-formamidinium hexafluorophosphate] at a temperature of the reaction mixture of about 20° C. and in the presence of a tertiary amine such as diisopropylethylamine.

The coupling conditions preferably use as reagent BOP (benzotriazol-1-yloxytris(dimethylamino)phosphonium hexafluorophosphate) in the presence of diisopropylethylamine in CH₂Cl₂.

The compounds of general formula (I) wherein X denotes a CH₂NH group may be obtained by a process comprising at least condensing a compound of general formula III as defined hereinbefore and a compound of general formula VI,

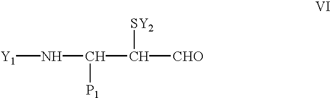

wherein Y₁, Y₂ and P₁ are as hereinbefore defined, then reducing the intermediate thus formed to produce the compound of general formula VII

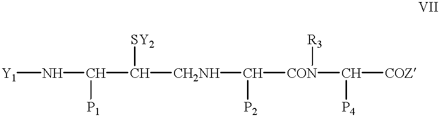

and, after deprotecting it, obtain the compound of general formula 1.

The dipeptide ester of general formula III has two asymmetric carbons. According to a preferred embodiment of the invention, the S configuration of each amino acid is favoured.

As for the compound of general formula IV, it has two asymmetric carbons C₂ and C₃. The stereochemistry of C₃ is defined by that of the amino acid used at the start of the synthesis of said compound IV according to one of the two synthesis diagrams defined hereinafter.

The intermediate may be reduced by any conventional method. Generally, it is reduced in situ by adding a sufficient quantity of an organic reducing agent such as a borohydride and more preferably sodium borohydride or cyanoborohydride.

As for the reactions of protection and deprotection these are within the capabilities of those skilled in the art.

As examples of the protecting groups which may be used within the scope of the present invention, a t. butyloxycarbonyl (t. Boc), benzyloxycarbonyl (Cbz) or fluorenyloxycarbonyl (Fmoc) group is particularly preferred for Y₁, and a p. methoxybenzyl or dimethoxybenzyl group is particularly preferred for Y₂.

Of course, these protecting groups are selected by considering the nature of the reaction to which the corresponding compounds are to be subjected and so as to effectively protect the functional groups in question during this reaction.

The reactions of deprotection are, of course, selected as a function of the nature of the protecting groups.

Thus, the compounds V and VII wherein Y₁, Y₂, P₁, P₂ and P₄ are preferably as hereinbefore defined are generally deprotected by treating with a mixture of TFA/anisol or BBr₃ in CH₂Cl₂ or HBr in acetic acid, or HF to produce compounds I (with X=CONH and X=CH₂NH, respectively).

However, when P₁ and/or P₄ contain a protected sulphonate group SO₃CH₂t.Bu, an additional step of deprotection by heating with tetramethylammonium chloride in DMF is necessary to produce the compounds I having groups R₁ and/or R₃ which have a free sulphonate, except in the case of deprotection by HF.

As for the compounds of general formula IV

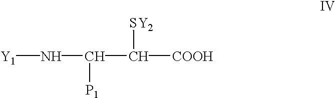

they may be obtained by two distinct methods of synthesis.

The first method of synthesis is illustrated by the following reaction plan.

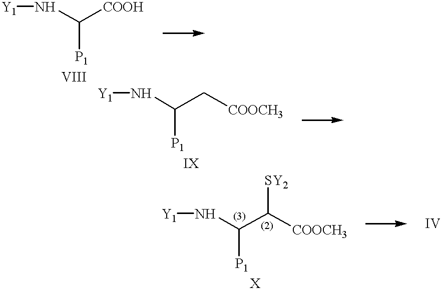

An α-amino acid protected on the N-terminal VIII is homologated by a method well known in the art into the β-amino ester IX. A reaction of sulphenylation by the reagents described in Shibata and al (Synlett. (1996)519-520; Tetrahedron 52(1996)12839-12852) or in Bischoff and al. (J. Org. Chem. 62(1997)48484850) in the presence of LDA or LiHMDS then leads to the derivative X which, after alkaline hydrolysis of the methyl ester, produces the compound IV.

The N-protected α-amino acids VIII are commercially available or may be prepared under enantioselective conditions using the method of Oppolzer (Tetrahedron Lett. 30(1989)6009-6010).

A second method of synthesis was used, illustrated by the following reaction plan.

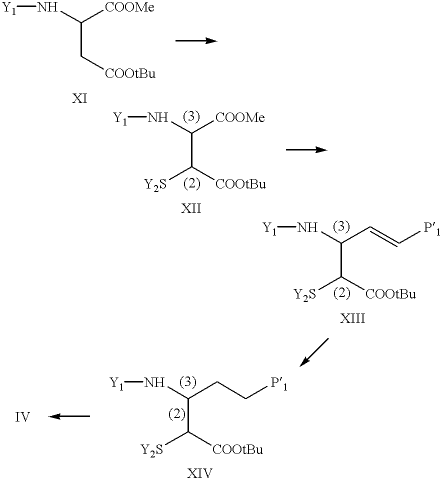

This second method of synthesis has the advantage of being diastereoselective. It comprises steps consisting of:

sulphenylating the protected diester N of formula XI of aspartic acid of the given configuration 2 into a compound XII whose configurations in positions 2 and 3 are identical but reversed compared with the configuration of the compound of formula XI,

reducing the ester α group into aldehyde,

condensing it in situ with an organic reagent so as to obtain a compound of general formula XIII of the same configuration and carrying a group P′₁ which is such that

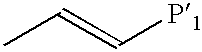

is a precursor of the group P₁,

reducing said compound of formula XIII into a compound XIV in the form of 2 diastereoisomers and

hydrolysing it in an acid medium to obtain the expected compound of formula IV.

The following may be proposed, in particular, as an example of this type of method of synthesis:

The N protected diester XI of aspartic acid of S configuration, for example, is sulphenylated as described in the previous method into compound XI of the 2R,3R configuration. The a ester group is reduced by dibal (diisobutyl aluminium hydride) into aiuminoxyacetal, then condensed in situ with a dialkylphosphonate or a phosphonium ylide, thus leading to the unsaturated compound XII of the same configuration. The reduction of XII by a copper hydride [(Ph₃P)CuH]₆ leads to XIII, predominantly of the same configuration (d.e. 85%). The reduction of the double bond by NaBH₄, by boron derivatives such as diborane in acetic acid, or cyclohexane in the presence of palladium leads to XIII in the form of 2 diastereoisomers. By acid hydrolysis, compound IV is obtained.

As stated previously, the compound IV has two asymmetric carbons, C₂ and C₃. The stereochemistry of C₃ is defined by that of the amino acid used at the start of synthesis (diagram 1 or diagram 2) as it is not modified during the different steps.

The reaction of sulphenylation is diastereoselective and is carried out in the anti position.

During the steps of reduction in a basic medium (XIII XIV) epimerisation on the carbon C₂ at the bottom of the sulphur leads to the missing stereoisomers which are separated by column chromatography after coupling of the dipeptide or by HPLC after coupling and partial deprotection.

In this way the four stereoisomers of the compound of general formula IV are obtained. According to a preferred embodiment of the invention, the formation of the 2R, 3R isomer is favoured.

The compounds claimed have proved capable of significantly and selectively inhibiting aminopeptidase A in relation to aminopeptidase N.

This selectivity factor is generally greater than 10² and more preferably 10³.

Consequently, the present invention also relates to the use of the compounds claimed as selective inhibitors of aminopeptidase A.

In this capacity, the compounds claimed may be used in therapy in all pathological processes induced by physiological substrates of aminopeptidase A such as the C-terminal octapeptide of cholecystokinine CCK8 and angiotensin II, the physiological roles of which were discussed earlier. They may thus be used to reduce food intake, modulate anxiety states or panic attacks or treat essential and secondary arterial hypertension, cardiac and renal failure, disorders of hydrodynamic homeostasis, myocardial infarction and proteinuria in diabetics.

For these purposes, the compounds claimed and the derivatives thereof may be used to prepare corresponding pharmaceutical compositions.

More particularly, the present invention relates, in another aspect, to a pharmaceutical composition containing as active ingredient at least one compound of general formula (I) or a derivative thereof.

Of course, this compound may be combined with at least one pharmaceutically acceptable carrier.

It is also possible to introduce two or more compounds of general formula I together into a single pharmaceutical composition.

These pharmaceutical compositions may be administered by oral, parenteral, sublingual, transdermal or topical route.

For administration by oral or sublingual route, plain or coated tablets, gelatine capsules, granules, optionally with delayed release, drops or liposomes may be used, in particular. For administration by intravenous, subcutaneous or intramuscular route, sterile or sterilisable solutions may be used, in particular, for venous perfusion, whereas conventional patches may be produced for administration by transdermal route.

The pharmaceutical compositions according to the present invention may be prepared by conventional methods well known in the field of pharmaceutical technology.

The active ingredient may be incorporated in the excipients normally used in these pharmaceutical compositions, such as talc, gum arabic, lactose, starch, magnesium stearate, aqueous or non-aqueous carriers, animal or vegetable fats, paraffin derivatives, glycols, various wetting, dispersing or emulsifying agents, preservatives, etc.

The quantity of active ingredient to be administered per day will depend of course on the nature of the therapeutic indication, the seriousness of the complaint to be treated as well as the patient's body weight and the route of administration.

For systemic administration, the overall dose in humans generally varies between 1 and 100 mg per day, for example 2 to 50 mg, and more appropriately 3 to 40 mg per day.

Single-dose forms for systemic administration will generally contain from 3 to 50 mg (i.e. 3, 5, 10, 20, 30, 40, and 50 mg de product). These single doses will normally be given one or more times a day, preferably one to three times a day.

For topical administration, the pharmaceutical compositions generally contain 0.0001 to 1% of active ingredient and preferably 0.01 to 5%.

The instant invention relates also to a method for the prevention or treatment of anxiety states or panic attacks, essential and secondary arterial hypertension, cardiac and renal failure, disorders of hydrodynamic homeostasis, myocardial infarct and proteinuria in diabetics, comprising administering to a patient in need of such treatment a therapeutically efficient amount of a compound of general formula (I).

The compounds described according to the invention may also be used in non-therapeutic applications, particularly in systems for diagnosing and measuring the expression of aminopeptidase A.

It is also claimed a method of diagnosis of anxiety states or panic attacks, essential and secondary arterial hypertension, cardiac and renal failure, disorders of hydrodynamic homeostasis, myocardial infarct and proteinuria in diabetics, wherein the aminopeptidase A is evaluated, in a biological sample of a patient to be tested, by using a claimed compound and is compared to the level present in normal subjects.

The present invention also relates to a diagnostic system for detecting and titrating aminopeptidase A, characterised in that it contains at least one compound of general formula I.

The following Examples, provided in a non-restrictive capacity, will demonstrate other advantages of the present invention.

### EXAMPLE 1

N-[[(2S,3R)- and (2R,3R)-3-amino-2-mercapto-5-sulphonate]pentanoyl]-L.Tyr-L.Sal-OH

STEP 1.

4-methyl and tert.butyl (2R,3R), 3-benzyloxycarbonylamino-2-(4-methoxybenzyl-sulphanyl)succinate.

Under argon, a 2.5 M solution of n-butyl lithium in n-hexane (56 ml; 140 mmol) is added dropwise at 0° C. to a solution of 1,1,1,3,3,3-hexamethyldisilazane (36 ml; 171 mmol) in dry tetrahydrofuran (375 ml) and the medium is shaken for ˜15 min at 0° C. The solution is cooled to −78° C. and the compound Z-L.Asp(OtBu)—OMe (20.7 g; 61 mmol) in dry THF (255 ml) is added dropwise. After stirring the reaction mixture for 2 h between −40° C. and −30° C., the medium is cooled to −78° C. and solid 4-methoxybenzyl and 2,4-dinitrophenyl disulphide (30 g; 85.4 mmol) is added. After 1 h at −78° C., the medium is hydrolysed with 1N HCl (200 ml) and the product is extracted with 500 ml of Et₂O. The organic phase is washed with 100 ml of citric acid, 100 ml of H₂O, 100 ml of saturated NaCl, dried over Na₂SO₄ and evaporated to dryness. The residue is taken up in cold Et₂O and the excess precipitate of sulphenylating agent is filtered. After evaporation to dryness the filtrate is purified by flash chromatography on silica gel (eluant cHex, AE, CH₂Cl₂ 8:1:1, R_{f}=0,19) giving yellow crystals, 20 g (68%) (ee: 95/5). HPLC C₁₈ Kromasil (5 μ, 100 Å) isocratic CH₃CN/H₂O (TFA) 75:25, t_{R}=9,9 min, P_{f}=71.6-73.3° C.

STEP 2.

tert.butyl (2R,3R)-3-benzyloxycarbonylamino-2-(4-methoxybenzyl sulphanyl)-5-(2,2-dimethylpropanoxysulphonyl)- pent-4-enoate.

A 2.5 M solution of n-butyl lithium in hexane (3.5 ml; 8.6 mmol) is added dropwise to a solution of neopentyl diethylphosphonomethane sulphonate (2.5 g ; 8.2 mmol) in dry THF (37 ml) at −78° C. under argon. The mixture is stirred for 30 min at −780C, then a solution of the compound of step 1 (2 g ; 4.1 mmol) in dry THF (4 ml) is added, followed, dropwise, by a 1.5 M solution of Dibal-H in toluene (5.3 ml; 8 mmol). After 4 hours' stirring at −78° C. and 1 hour's stirring at ambient temperature, the reaction mixture is hydrolysed with H₂O (8 ml) and 2N HCl (8 ml). The aqueous phase is then extracted with 160 ml of EA. The organic phase is washed with saturated NaCl, dried over Na₂SO₄ and the solvent is evaporated to dryness. The pure product is obtained after flash chromatography (eluant cHex, EA, CH₂CI₂ 7:1.5:1.5, R_{f}=0,39) in the form of a yellow oil, 1.6 g (68%). HPLC C₁₈ Kromasil (5 μ, 100 Å) isocratic CH₃CN/H₂O (TFA) 75:25 t_{R}=17,1 min. SM (Electrospray): 629,8=Mna⁺.

STEP 3.

Procedure 1.

tert.butyl (2RS,3R), 3-benzyloxycarbonylamino-2-(4-methoxybenzyl sulphanyl)-5-(2,2-dimethylpropanoxysulphonyl)pentanoate.

The ethylene compound obtained in step 2 (10 g 16.4 mmol) in EtOH (164 ml) is reduced by NaBH₄ (996 mg; 26.3 mmol) for 3 h at ambient temperature. After a conventional treatment, the product is obtained in the form of an orange oil, 10 g (99%). R_{f} (cHex, CH₂Cl₂, EA 6:2:2)=0.50. It has total epimerisation at the C₂. HPLC C₁₈ Kromasil (5 μ, 100 Å) isocratic CH₃CN/H₂O (TFA) 75:25 t_{R}=15.9 min and 16.6 min.

Procedure 2.

tert.butyl (2R,3R), 3-benzyloxycarbonylamino-2-(4-methoxybenzyl sulphanyl)-5-(2,2-dimethylpropanoxysulphonyl)pentanoate.

A solution of the previous ethylene compound (100 mg; 0.16 mmol) in benzene (2.5 ml) is degassed under argon for 15 minutes. To this solution are added, successively, at ambient temperature, 60 μl (3.3 mmol) of water, then 120 mg (0.06 mmol) of [(Ph₃P)CuH]₆. The reaction medium is stirred for 3 hours at ambient temperature then hydrolysed with a solution of NH₄Cl, and extracted with EA. The organic phase is washed with saturated NaCl, dried over Na₂SO₄ and evaporated to dryness, giving the product (80 mg; 80%) in the form of a yellow oil having an enantiomeric excess of 85:15. HPLC C₁₈ Kromasil (5 μ, 100 Å) socratic CH₃CN/H₂O (TFA) 75:25 t_{R}=16.6 min.

STEP 4.

(2RS, 3R), 3-benzyloxycarbonylamino-2-(4-methoxybenzyl sulphanyl)-5-(2,2-dimethylpropanoxysulphonyl) pentanoic acid.

A solution of the compound of step 3 (10 g; 16.4 mmol) in CH₂Cl₂ (13 ml) is treated with anisol (630 μl; 5% vol.) and TFA (12.6 ml; 164 mmol) and after 3 hours' stirring at ambient temperature leads to the expected acid, which is chromatographed (silica gel, flash, eluant cHex, Et₂O, HCOOH 4:6:0.1, R_{f}=0.40) giving a red foam, 6.1 g (69%). HPLG C₁₈ Kromasil (5 μ, 100 Å) isocratic CH₃CN/H₂O (TFA) 75:25 t_{R}=5.6 min.

STEP 5.

N-[(2S,3R) and (2R,3R)-3-benzyloxycarbonylamino-5-[2,2-dimethylpropanoxy-sulphonyl]-2-[4-methoxybenzylsulphanyl]pentanoyl]-L.Tyr(t.Bu)-L.Sal(OCH₂tBu)OtBu

The acid is coupled in the solid phase with the dipeptide H-Tyr(tBu)-Sal(OCH₂tBu) grafted onto a Wang-type resin using the coupling agent BOP and diisopropylethylamine.

STEP 6.

N-[(2R,3R) and (2S,3R)-3-benzyloxycarbonylamino-5-[2,2-dimethylpropanoxy-sulphonyl]-2-[4-methoxybenzylsulphanyl]pentanoyl]-L.Tyr-L.Sal-OH.

Peptidyl resin in CH₂Cl₂ (2 ml) is treated at 0° C. with 60 μl of anisol (5% vol.) and trifluoroacetic acid (1.2 ml; 15 mmol) and the reaction mixture is stirred for 3.5 hours at ambient temperature. After evaporation to dryness, the residue is taken up in cold Et₂O and the precipitate obtained is triturated in the same solvent. The product is recovered, after decanting the ethereal phase, in the form of a white powder.

STEP 7.

N-[[(2RS,3R), 3-amino-2-mercapto-5-sulphonate]pentanoyl]-L.Tyr-L.Sal-OH.

The pseudo-tripeptide of step 6 (0.43 mmol) is treated with 500 μl of meta-cresol distilled beforehand, then, at −78° C., with 10 ml of liquid hydrogen fluoride. After 1 hour's stirring at 0° C., the HF is evaporated in vacuo. The residue is taken up in 2×1 ml of TFA and the product is obtained by precipitation in cold Et₂O/n-hexane and centrifugation. The compound is finally purified by semi-preparative HPLC with C₁₈ Kromasil (10 μ, 100 Å) on a CH₃CN/H₂O (TFA) gradient from 10 to 60%, t_{R}−4.1 min, to yield a white powder (20 mg). SM (Electrospray): 544.6=MH⁺, 566.6=Mna⁺.

### EXAMPLE 2

N-[[(2S,3R)- and (2R,3R)-3-amino-2-mercapto-5-sulphonate]pentanoyl]-L.Tyr-L.hSal-OH

In accordance with the process used in Example 1 with the dipeptide H-Tyr(tBu)-hSal(OCH₂tBu) grafted onto a Wang resin, the two stereoisomers separated are obtained in the form of 2 white powders (14 and 17 mg). HPLC C₈ Kromasil (5 μ, 100 Å) gradient CH₃CN/H₂O (TFA) from 10 to 60%, t_{R}=2.8 min and 3.4 min. SM (Electrospray): 557.9=MH⁺, 579.9=Mna⁺.

### EXAMPLE 3

N-[[(2S ,3R)- and (2R,3R)-3-amino-5-carboxy-2-mercapto]pentanoyl]-L.IIe-L.(3R)(3-COOH)Pro-OH

This compound is obtained using the procedure described for Example 1, using Z-L-Asp (OtBu)OMe in step 1, IIe-(3R)(3-CO₂CH₂Ph)ProOCH₂Ph dipeptide in step 5 and omitting step 6.

3A: [[(2S ,3R)-3-amino-5-carboxy-2-mercapto]pentanoyl]-LIIe-L.(3R)(3-COOH)Pro-OH

White powder (5 mg). HPLC C₁₈ Kromasil (5 μ, 100 Å)CH₃CN/H₂O (TFA) 15/85, t_{R}=8,5 min. SM (Electrospray): 448.0=MH⁺.

3B: [[(2R,3R), 3-amino-5-carboxy-2-mercapto]-pentanoyl]-L.IIe-L.(3R)(3-COOH)Pro-OH

White powder (10 mg). HPLC C₁₈ Kromasil (5 μ, 100 Å) CH₃CN/H₂O (TFA) 15/85, t_{R}=6.0 min. SM (Electrospray): 448.0=MH⁺.

### EXAMPLE 4

N-[[(2S ,3R)- and (2R,3R)-3-amino-5-phosphonate-2-mercapto]pentanoyl]-L.IIe-L.Glu-OH

This compound is obtained using the procedure described for Example 1, using tetrabenzyl diphosphoryl methylene in step 2 and IIe-Glu(OtBu)OtBu dipeptide in step 5.

4A: [[(2S ,3R)-3-amino-5-phosphonate-2-mercapto]-pentanoyl]-L.IIe-L.Glu-OH

White powder (23 mg). HPLC C₁₈ Kromasil (5 μ, 100 Å) CH₃CN/H₂O (TFA) 15/85, t_{R}=6.97 min. SM (Electrospray) 472.4=MH⁺.

4B: [[(2R,3R)-3-amino-5-phosphonate-2-mercapto]-pentanoyl]-L.IIe-L.Glu-OH

White powder (33 mg). HPLC C₁₈ Kromasil (5 μ, 100 Å) CH₃CN/H₂O (TFA) 15/85, t_{R}=4.6 min. SM (Electrospray) 472.5=MH⁺.

### EXAMPLE 5

N-[[2S,3R) and (2R,3R), 3-amino-2-mercapto-5-sulphonate]pentanoyl]-L.IIe-L.Sal-OH

This compound is obtained using the procedure described for Example 1, using the dipeptide IIe-Sal(CH₂tBu)OH in step 5.

5A: [[(2S,3R)-3-amino-2-mercapto-5-sulphonate]-pentanoyl]-L.IIe-L.Sal-OH

White powder (4 mg). HPLC C₁₈ Kromasil (5 μ, 100 Å) CH₃CN/H₂O (TFA) 15/85, t_{R}=4.1 min. SM: 493.9=MH⁺.

5B: [[(2R,3R), 3-amino-2-mercapto-5-sulphonate]-pentanoyl]- L.IIe-L.Sal-OH

White powder (13 mg). HPLC C18 Kromasil (5 μ, 100 Å) CH₃CN/H₂O (TFA) 15/85, t_{R}=2.4 min.

### EXAMPLE 6

N-[[(2S,3R)- and (2R,3R)-3-amino-2-mercapto-5-sulphonate]pentanoyl]-L.IIe-L.(3R)(3-COOH)Pro-OH

This compound is obtained using the procedure described for Example 1, using the dipeptide IIe-(3R)(3-CO₂CH₂Ph)Pro OCH₂Ph in step 5 and omitting step 6.

6A: [[(2S,3R), 3-amino, 2-mercapto-5, sulphonate]-pentanoyi]- L.IIe-L.(3R)(3—COOH)Pro-OH

White powder (19 mg). HPLC C₁₈ Kromasil (5 μ, 100 Å) CH₃CN/H₂O (TFA) 15/85, t_{R}=9.9 min. SM 484.1=MH⁺, 506.1=Mna⁺.

6B: [[(2R,3R), 3-amino-2-mercapto-5-sulphonate]-pentanoyl]- L.IIe-L.(3 R)(3—COOH)Pro-OH

White powder (33 mg). HPLC C₁₈ Kromasil (5 μ, 100 Å) CH₃CN/H₂O (TFA) 15/85, t_{R}=3.6 min. SM: 484.1=MH⁺.

### EXAMPLE 7

N-[[(2S,3R)- and (2R,3R)-3-amino-2-mercapto-5-sulphonate]pentanoyl]-L.IIe-L.(3S)(3-COOH)Pro-OH

This compound is obtained using the procedure described for Example 1, using the dipeptide IIe-(3S)(3- CO₂CH₂Ph)ProOCH₂Ph in step 5 and omitting step 6.

7A: [[(2S,3R), 3-amino-2-mercapto-5-sulphonate]-pentanoyl]-L.IIe-L.(3S)(3-COOH)Pro-OH

White powder (4 mg). HPLC C₁₈ Kromasil (5 μ, 100 Å) CH₃CN/H₂O (TFA) 15/85, t_{R}=14.9 min. SM 484.5=MH⁺, 522.5=MK⁺.

7B: [[(2R,3 R), 3-amino-2-mercapto-5-sulphonate]-pentanoyl]-L.IIe-L.(3S)(3-COOH)Pro-OH

White powder (6 mg). HPLC C₁₈ Kromasil (5 μ, 100 Å) CH₃CN/H₂O (TFA) 15/85, t_{R}=4.7 min. SM: 484.5=MH⁺, 522.3=MK⁺.

### EXAMPLE 8

N-[[(2S,3R)- and (2R,3R)-3-amino-2-mercapto-5-sulphonate]pentanoyl]-L.IIe-L.Glu-NH₂

By following steps 1 to 7 described in Example 1 but coupling with the dipeptide H-IIe-Glu(OtBu)-NH₂, the two stereoisomers of the desired compound are obtained, in the form of 2 white powders (20 mg and 10 mg). HPLC C₁₈ Kromasil (5 μ, 100 Å) isocratic CH₃CN/H₂O (TFA) 15/85, t_{R}=3.5 min and 4.9 min. SM (Electrospray):470.9=MH⁺.

Benzyl diethyl phosphoryl acetate or benzyl (diethoxy-phosphoryl)-acetate.

This reagent is synthesised according to the procedure described by Jennings and coil. (1992).

Tetrabenzyl diphosphoryl methylene or dibenzyl (dibenzyloxy-phosphoryl-methyl)-phosphonate.

This reagent is synthesised according to the procedure described by Saady and coil. (1995).

### EXAMPLE 9

In vitro Study of the Inhibiting Power of the Synthesised Compounds on Aminopeptidase a and Aminopeptidase N.

a) In vitro study of the inhibiting power of the synthesised compounds on aminopeptidase A.

1°) Purification of the Enzyme.

Aminopeptidase A is obtained from rabbit kidney as described in “Neuropeptides 16 (1990)163-168”. The synthetic substrate used is L-glutamyl-β-naphthylamide (Glu NA, Km=130 μM). The purified enzyme has a specific activity with regard to Glu NA of 100 μmol.mL⁻¹.li⁻¹.

2°)Titration Method.

The process described by Goldbarg “Cancer 11 (1958)283-291” was scaled down for the microtitre plate.

The APA is incubated for 1 h at 37° C. in the presence of 200 μM Glu NA and in the presence of variable concentrations of inhibitors in Tris HCl buffer (50 mM). pH=7.4, 4 mM of CaCl₂ (final volume 100 μl). The reaction is stopped by the addition of 10 μl of 3N HCl. Then 25 μl of NaNO₂ (87 mM) are added to each well followed, 3 min later, by 50 μl of ammonium sulphamate (0.13 M). Finally, after 5 min, 25 μl of a 23 mM solution of 1-naphthylethylene diamine are added at 37° C. The absorption at 560 nm is measured and compared with that of a standard calibration range of 2-naphthylamine.

b)) In vitro study of the inhibiting power of the synthesised compounds on aminopeptidase N.

1°) Source of the Enzyme.

Aminopeptidase N purified from pig's kidney is marketed by Böehringer Mannheim (Meylan, France).

2°) Titration Method.

The APN is incubated for 10 min at 37° C. in the absence or in the presence of increasing concentrations of inhibitors in a total volume of 100 μl in Tris HCl buffer (50 mM, pH =7.4. 200 μM of Ala-Na are added and the whole thing is incubated for 30 min at 37° C. The reaction is stopped by the addition of 10 μl of CH₃COO—Na 1M (pH=4.2). The absorption is measured at 400 nM and compared with that of a standard range of 2-naphthyiamine.

| | | | | | | | | Ki (M) | Ki (M) |
|---|---|---|---|---|---|---|---|---|---|
| | R₁ | X | R₂ | R₃ | R₄ | Z | Example | APA | APN |
| Ex 1 | (CH₂)₂—SO₃⁻ | CO | CH₂-p(C₆H₄)—OH | H | CH₂SO₃H | OH | 1A + B | 3, 7.10⁻⁸ | 1, 0.10⁻⁵ |
| Ex 2 | (CH₂)₂—SO₃⁻ | CO | CH₂-p(C₆H₄)—OH | H | (CH₂)₂SO₃H | OH | 2A | 9, 3.10⁻⁹ | 4, 6.10⁻⁶ |
| | | | | | | | 2B | 7, 3.10⁻⁸ | 7, 5.10⁻⁶ |
| Ex 3 | (CH₂)₂—CO₂⁻ | CO | CH(CH₃)CH₂CH₃ | (3R)(3-CO₂H)Pro | | OH | 3A | 0, 72.10⁻⁹ | >5.10⁻⁶ |
| | | | | | | | 3B | 6, 5.10⁻⁸ | >5.10⁻⁶ |
| Ex 4 | (CH₂)₂—P(O)(OH)—O⁻ | CO | CH(CH₃)CH₂CH₃ | H | (CH₂)₂—CO₂H | OH | 4A | 1, 4.10⁻⁸ | >5.10⁻⁶ |
| | | | | | | | 4B | 9, 5.10⁻⁷ | >5.10⁻⁶ |
| Ex 5 | (CH₂)₂—SO₃⁻ | CO | CH(CH₃)CH₂CH₃ | H | CH₂-SO₃H | OH | 5A | 3, 6,10⁻⁹ | >5.10⁻⁶ |
| | | | | | | | 5B | 8, 3.10⁻⁸ | >5.10⁻⁶ |
| Ex 6 | (CH₂)₂—SO₃⁻ | CO | CH(CH₃)CH₂CH₃ | (3R)(3-CO₂H)Pro | | OH | 6A | 0, 87.10⁻⁹ | 1, 64.10⁻⁵ |
| | | | | | | | 6B | 1, 1.10⁻⁷ | >10⁻⁵ |
| Ex 7 | (CH₂)₂—SO₃⁻ | CO | CH(CH₃)CH₂CH₃ | (3S)(3-CO₂H)Pro | | OH | 7A | 3, 8.10⁻⁹ | 4, 9.10⁻⁶ |
| | | | | | | | 7B | 2, 4.10⁻⁷ | >10⁻⁵ |
| Ex 8 | (CH₂)₂—SO₃⁻ | CO | CH(CH₃)CH₂CH₃ | H | (CH₂)₂—CO₂H | NH₂ | 8A | 2, 5.10⁻⁸ | >5.10⁻⁵ |
| | | | | | | | 8B | 6, 3.10⁻⁷ | >5.10⁻⁵ |

### Attachment A

### Clean New/Replacement Paragraphs

At the following location, add the new section indicated below.

Page 1, before the first line between the Title and the second line, insert the following section.

Related Applications

This application is a continuation-in-part of international application number PCT/FR00/00112, filed Jan. 19, 2000.

1. A compound of general formula I: 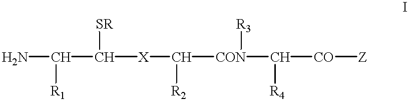
wherein:
R₁ denotes an alkyl, alkenyl or alkynyl chain, or a cycloalkyl or (cycloalkyl)alkyl group substituted by at least one
—COOH group, optionally esterified by an alkyl group comprising 2 to 12 carbon atoms,
SO₃H group, optionally protected by a pentyl group,
PO₃H₂ group, optionally substituted by a (—CH₂CH₂SCOR₅) group, with R₅ representing a C₁-C₄ alkyl group, a phenyl or benzyl group, or
tetrazolyl group.
R₂ denotes an alkyl chain, or an aryl, arylalkyl, cycloalkyl, (cycloalkyl)alkyl, (heteroaryl)alkyl group which may or may not be substituted by at least one OH, OR, SR′, NH₂, NHR′, guanidinyl, COOH or CONH₂ group, or a halogen atom selected from among F, Cl, Br or I with R′ representing a straight-chain or branched C₁₋₄ alkyl group.
R₃ denotes a hydrogen atom or a methyl group,
R₄ denotes
an alkyl chain, an aryl, arylalkyl, cycloalkyl, (cycloalkyl)alkyl, (heteroaryl)alkyl, heterocycloalkyl or (heterocycloalkyl)alkyl group substituted by at least one CONH₂, SO₃H, SO₂NH₂, PO₃H₂ or tetrazolyl group, with the groups SO₃H and PO₃H₂ optionally protected,
a C₂₋₆ alkyl chain, an aryl, arylalkyl, cycloalkyl, (cycloalkyl)alkyl, (heteroaryl)alkyl, heterocycloalkyl or (heterocycloalkyl)alkyl group substituted by at least one CO₂H group optionally protected, or
R₃ and R₄ may together constitute a 5- or 6-membered heterocyclic compound, substituted by at least one CO₂H, CONH₂, SO₃H, SO₂NH₂ or PO₃H₂ group with the groups CO₂H, SO₃H and PO₃H₂ optionally protected,
X denotes a group CONH or CH₂NH,
Z denotes a group OH, OCH₂-C₆H₅ or NR″R′″ wherein R″ and R′″ independently of one another may denote a hydrogen atom or an alkyl, aryl or arylalkyl group, where R″ and R′″ may constitute, together with the nitrogen atom, a 5- or 6-membered heterocycle possibly having a second heteroatom selected from among O, S and N, and
R denotes a hydrogen atom or a group of formula II 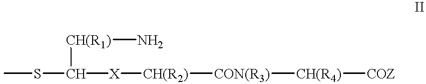
corresponding to the symmetric disulphide of the inhibitor wherein R₁, R₂, R₃, R4, X and Z are as hereinbefore defined, and the derivatives thereof.

2. The compound according to claim 1, wherein
R₄ denotes an alkyl chain, an aryl, arylalkyl, cycloalkyl, (cycloalkyl)alkyl, (heteroaryl)alkyl, heterocycloalkyl or (heterocycloalkyl)alkyl group substituted by at least one CONH2, SO₃H, SO₂NH₂, PO₃H₂ or tetrazolyl group, with the groups SO₃H and PO₃H₂ optionally being protected as described above, or
R₄ constitutes with R₃ a 5- or 6-membered heterocyclic compound, substituted by at least one CO₂H, CONH₂, SO₃H, SO₂NH₂ or PO₃H₂ group with the groups CO₂H, SO₃H and PO₃H₂ optionally being protected.

3. The compound according to claim 1, wherein R₄ and R₃ together constitute a 5- or 6-membered, heterocyclic compound substituted by at least one group CO₂H, CONH₂, SO₃H, SO₂NH₂ or PO₃H₂ with the groups CO₂H, SO₃H and PO₃H₂ optionally being protected.

4. The compound according to claim 1, wherein X denotes a CONH function.

5. The compound according to claim 1, wherein R₂ denotes an optionally substituted alkyl or arylalkyl chain.

6. The compound according to claim 1, which is selected from the group consisting in
N-[[(2S,3R)- and (2R,3R)-3-amino-2-mercapto-5-sulphonate]pentanoyl]-L.Tyr-L.Sal-OH;
N-[[(2S,3R)- and (2R,3R)-3-amino-2-mercapto-5-sulphonate]pentanoyl]-L.Tyr-L.hSal-OH
N-[[(2S,3R)- and (2R,3R)-3-amino-5-carboxy-2-mercapto]pentanoyl]-L.IIe-L.(3R)(3-COOH)Pro-OH
N-[[(2S,3R)- and (2R,3R)-3-amino-5-phosphonate-2-mercapto]pentanoyl]-L.IIe-L.Glu-OH;
the N-[[2S,3R) and (2R,3R), 3-amino-2-mercapto-5-sulphonate]pentanoyl]-L.IIe-L.Sal-OH;
N-[[(2S,3R)- and (2R,3R)-3-amino-2-mercapto-5-sulphonate]pentanoyl]-L.IIe-L.(3R)(3-COOH)Pro-OH;
N-[[(2S,3R)- and (2R,3R)-3-amino-2-mercapto-5-sulphonate]pentanoyl]-L.IIe-L.(3S)(3-COOH)Pro-OH; and
N-[[(2S,3R)- and (2R,3R)-3-amino-2-mercapto-5-sulphonate]pentanoyl]-L.IIe-L.Glu-NH₂.

7. A process for preparing a compound of general formula I 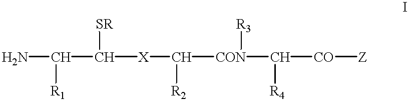
wherein
R₁ denotes an alkyl, alkenyl or alkynyl chain, or a cycloalkyl or (cycloalkyl)alkyl group substituted by at least one
—COOH group, optionally esterified by an alkyl group comprising 2 to 12 carbon atoms,
SO₃H group, optionally protected by a pentyl group,
PO₃H₂ group, optionally substituted by a (—CH₂CH₂SCOR₅) group, with R₅ representing a C₁-C₄ alkyl group, a phenyl or benzyl group, or
tetrazolyl group.
R₂ denotes an alkyl chain, or an aryl, arylalkyl, cycloalkyl, (cycloalkyl)alkyl, (heteroaryl)alkyl group which may or may not be substituted by at least one OH, OR, SR′, NH₂, NHR′, guanidinyl, COOH or CONH₂ group, or a halogen atom selected from among F, Cl, Br or I with R′ representing a straight-chain or branched C₁₄ alkyl group.
R₃ denotes a hydrogen atom or a methyl group,
R₄ denotes
an alkyl chain, an aryl, arylalkyl, cycloalkyl, (cycloalkyl)alkyl, (heteroaryl)alkyl, heterocycloalkyl or (heterocycloalkyl)alkyl group substituted by at least one CONH₂, SO₃H, SO₂NH₂, PO₃H₂ or tetrazolyl group, with the groups SO₃H and PO₃H₂ optionally protected,
a C₂₋₆ alkyl chain, an aryl, arylalkyl, cycloalkyl, (cycloalkyl)alkyl, (heteroaryl)alkyl, heterocycloalkyl or (heterocycloalkyl)alkyl group substituted by at least one CO₂H group optionally protected, or
R₃ and R₄ may together constitute a 5- or 6-membered heterocyclic compound, substituted by at least one CO₂H, CONH₂, SO₃H, SO₂NH₂ or PO₃H₂ group with the groups CO₂H, SO₃H and PO₃H₂ optionally protected,
X denotes a group CONH,
Z denotes a group OH, OCH₂-C₆H₅ or NR″R′″ wherein R″ and R′″ independently of one another may denote a hydrogen atom or an alkyl, aryl or arylalkyl group, where R″ and R′″ may constitute, together with the nitrogen atom, a 5- or 6-membered heterocycle possibly having a second heteroatom selected from among O, S and N, and
R denotes a hydrogen atom or a group of formula II 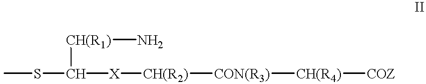
corresponding to the symmetric disuiphide of the inhibitor wherein R₁, R₂, R₃, R₄, X and Z are as hereinbefore defined,
which involves at least coupling an ester dipeptide of general formula III 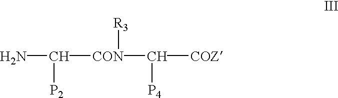
wherein
P₂ and P₄ correspond to protected forms of R₂ and R₄,
Z′ denotes an OC(CH₃)₃, OCH₂-C₆H₅ or NR″R′″ group wherein R″ and R′″ independently of one another may denote a hydrogen atom or an alkyl, aryl or arylalkyl group, while R″ and R′″ may constitute, together with the nitrogen atom, a 5- or 6-membered heterocycle possibly having a second heteroatom selected from among O, S and N, with a compound of general formula IV 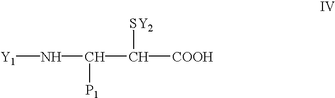
wherein:
Y₁ denotes a protecting group
Y₂ denotes a protecting group and
P₁ denotes a protected form of R₁, under conditions suitable to produce compound V 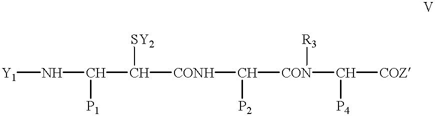
and deprotecting it for obtaining said compound of general formula 1.

8. The process according to claim 7, wherein the coupling reaction is carried out in an organic solvent in the presence of a coupling agent and a tertiary amine and at a temperature of the order of 20° C.

9. A process according to claim 7, wherein the two asymmetric carbons of the dipeptide ester of general formula III have an S configuration.

10. A process for preparing a compound of general formula I 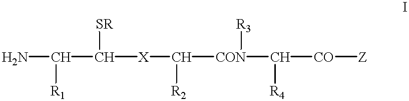
wherein:
R₁ denotes an alkyl, alkenyl or alkynyl chain, or a cycloalkyl or (cycloalkyl)alkyl group substituted by at least one
—COOH group, optionally esterified by an alkyl group comprising 2 to 12 carbon atoms,
SO₃H group, optionally protected by a pentyl group,
PO₃H₂ group, optionally substituted by a (—CH₂CH₂SCOR₅) group, with R₅ representing a C₁-C₄ alkyl group, a phenyl or benzyl group, or
tetrazolyl group.
R₂ denotes an alkyl chain, or an aryl, arylalkyl, cycloalkyl, (cycloalkyl)alkyl, (heteroaryl)alkyl group which may or may not be substituted by at least one OH, OR, SR′, NH₂, NHR′, guanidinyl, COOH or CONH₂ group, or a halogen atom selected from among F, Cl, Br or I with R′ representing a straight-chain or branched C₁₋₄ alkyl group.
R₃ denotes a hydrogen atom or a methyl group,
R₄ denotes
an alkyl chain, an aryl, arylalkyl, cycloalkyl, (cycloalkyl)alkyl, (heteroaryl)alkyl, heterocycloalkyl or (heterocycloalkyl)alkyl group substituted by at least one CONH₂, SO₃H, SO₂NH₂, PO₃H₂ or tetrazolyl group, with the groups SO₃H and PO₃H₂ optionally protected,
a C₂₋₆ alkyl chain, an aryl, arylalkyl, cycloalkyl, (cycloalkyl)alkyl, (heteroaryl)alkyl, heterocycloalkyl or (heterocycloalkyl)alkyl group substituted by at least one CO₂H group optionally protected, or
R₃ and R₄ may together constitute a 5- or 6-membered heterocyclic compound, substituted by at least one CO₂H, CONH₂, SO₃H, SO₂NH₂ or PO₃H₂ group with the groups CO₂H, SO₃H and PO₃H₂ optionally protected,
X denotes a CH₂-NH group,
Z denotes a group OH, OCH₂-C₆H₅ or NR″R′″ wherein R″ and R′″ independently of one another may denote a hydrogen atom or an alkyl, aryl or arylalkyl group, where R″ and R′″ may constitute, together with the nitrogen atom, a 5- or 6-membered heterocycle possibly having a second heteroatom selected from among O, S and N, and
R denotes a hydrogen atom or a group of formula II 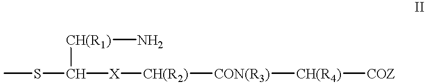
corresponding to the symmetric disulphide of the inhibitor wherein R₁, R₂, R₃, R₄, X and Z are as hereinbefore defined, which involves at least:
condensing a compound of general formula III 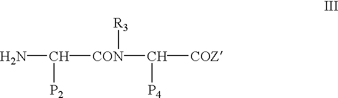
wherein Z′ denotes an OC(CH₃)₃, OCH₂-C₆H₅ or NR″R′″ group with R″ and R′″ independently of one another may denoting a hydrogen atom or an alkyl, aryl or arylalkyl group, where R″ and R′″ may, together with the nitrogen atom, constitute a 5- or 6-membered heterocycle possibly having a second heteroatom selected from among O, S and N,
with a compound of general formula VI, 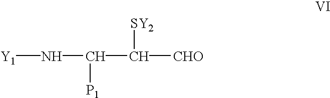
wherein:
Y₁ denotes a protecting group
Y₂ denotes a protecting group and
P₁ denotes a protected form of R₁,
reducing the intermediate thus formed to produce the compound of general formula VII 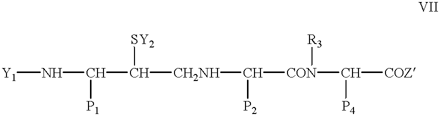
by deprotecting the so-obtained compound and obtaining said compound of general formula I.

11. A process according to claim 9, wherein the two asymmetric carbons of the dipeptide ester of general formula III have an S configuration.

12. Use of a compound of formula (I): 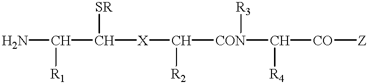
wherein:
R₁ denotes an alkyl, alkenyl or alkynyl chain, or a cycloalkyl or (cycloalkyl)alkyl group substituted by at least one
—COOH group, optionally esterified by an alkyl group comprising 2 to 12 carbon atoms,
SO₃H group, optionally protected by a pentyl group,
PO₃H₂ group, optionally substituted by a (—CH₂CH₂SCOR₅) group, with R₅ representing a C₁-C₄ alkyl group, a phenyl or benzyl group, or
tetrazolyl group.
R₂ denotes an alkyl chain, or an aryl, arylalkyl, cycloalkyl, (cycloalkyl)alkyl, (heteroaryl)alkyl group which may or may not be substituted by at least one OH, OR, SR′, NH₂, NHR′, guanidinyl, COOH or CONH₂ group, or a halogen atom selected from among F, Cl, Br or I with R′ representing a straight-chain or branched C₁₋₄ alkyl group.
R₃ denotes a hydrogen atom or a methyl group,
R₄ denotes
an alkyl chain, an aryl, arylalkyl, cycloalkyl, (cycloalkyl)alkyl, (heteroaryl)alkyl, heterocycloalkyl or (heterocycloalkyl)alkyl group substituted by at least one CONH₂, SO₃H, SO₂NH₂, PO₃H₂ or tetrazolyl group, with the groups SO₃H and PO₃H₂ optionally protected,
a C₂₋₆ alkyl chain, an aryl, arylalkyl, cycloalkyl, (cycloalkyl)alkyl, (heteroaryl)alkyl, heterocycloalkyl or (heterocycloalkyl)alkyl group substituted by at least one CO₂H group optionally protected, or
R₃ and R₄ may together constitute a 5- or 6-membered heterocyclic compound, substituted by at least one CO₂H, CONH₂, SO₃H, SO₂NH₂ or PO₃H₂ group with the groups CO₂H, SO₃H and PO₃H₂ optionally protected,
X denotes a group CONH or CH₂NH,
Z denotes a group OH, OCH₂-C₆H₅ or NR″R′″ wherein R″ and R′″ independently of one another may denote a hydrogen atom or an alkyl, aryl or arylalkyl group, where R″ and R′″ may constitute, together with the nitrogen atom, a 5- or 6-membered heterocycle possibly having a second heteroatom selected from among 0, S and N, and
R denotes a hydrogen atom or a group of formula II 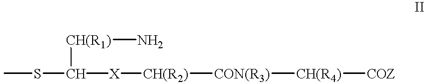
corresponding to the symmetric disulphide of the inhibitor wherein R₁, R₂, R₃, R₄, X and Z are as hereinbefore defined,
and the derivatives thereof,
as a selective inhibitor of aminopeptidase A.

13. Use according to claim 12 for preparing a medicament intended to reduce food intake, modulate anxiety states or panic attacks or treat essential and secondary arterial hypertension, cardiac and renal failure, disorders of hydrodynamic homeostasis, myocardial infarct and proteinuria in diabetics.

14. Pharmaceutical composition containing as active ingredient at least one compound of general formula (I): 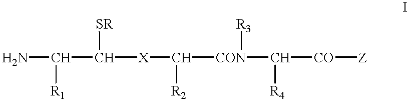
wherein
R₁ denotes an alkyl, alkenyl or alkynyl chain, or a cycloalkyl or (cycloalkyl)alkyl group substituted by at least one
—COOH group, optionally esterified by an alkyl group comprising 2 to 12 carbon atoms,
SO₃H group, optionally protected by a pentyl group,
PO₃H₂ group, optionally substituted by a (—CH₂CH₂SCOR₅) group, with R₅ representing a C₁-C₄ alkyl group, a phenyl or benzyl group, or
tetrazolyl group.
R₂ denotes an alkyl chain, or an aryl, arylalkyl, cycloalkyl, (cycloalkyl)alkyl, (heteroaryl)alkyl group which may or may not be substituted by at least one OH, OR, SR′, NH₂, NHR′, guanidinyl, COOH or CONH₂ group, or a halogen atom selected from among F, Cl, Br or I with R′ representing a straight-chain or branched C₁₋₄ alkyl group.
R₃ denotes a hydrogen atom or a methyl group,
R₄ denotes
an alkyl chain, an aryl, arylalkyl, cycloalkyl, (cycloalkyl)alkyl, (heteroaryl)alkyl, heterocycloalkyl or (heterocycloalkyl)alkyl group substituted by at least one CONH₂, SO₃H, SO₂NH₂, PO₃H₂ or tetrazolyl group, with the groups SO₃H and PO₃H₂ optionally protected,
a C₂₋₆ alkyl chain, an aryl, arylalkyl, cycloalkyl, (cycloalkyl)alkyl, (heteroaryl)alkyl, heterocycloalkyl or (heterocycloalkyl)alkyl group substituted by at least one CO₂H group optionally protected, or
R₃ and R₄ may together constitute a 5- or 6-membered heterocyclic compound, substituted by at least one CO₂H, CONH₂, SO₃H, SO₂NH₂ or PO₃H₂ group with the groups CO₂H, SO₃H and PO₃H₂ optionally protected,
X denotes a group CONH or CH₂NH,
Z denotes a group OH, OCH₂-C₆H₅ or NR″R′″ wherein R″ and R′″ independently of one another may denote a hydrogen atom or an alkyl, aryl or arylalkyl group, where R″ and R′″ may constitute, together with the nitrogen atom, a 5- or 6-membered heterocycle possibly having a second heteroatom selected from among O, S and N, and
R denotes a hydrogen atom or a group of formula II 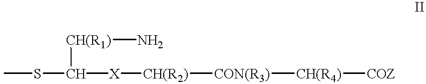
corresponding to the symmetric disulphide of the inhibitor wherein R₁, R₂, R₃, R₄, X and Z are as hereinbefore defined,
and the derivatives thereof.

15. Diagnostic system for detecting and titrating aminopeptidase A, characterised in that it contains at least one compound of general formula (I): 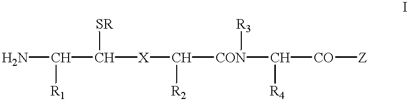
wherein:
R₁ denotes an alkyl, alkenyl or alkynyl chain, or a cycloalkyl or (cycloalkyl)alkyl group substituted by at least one
—COOH group, optionally esterified by an alkyl group comprising 2 to 12 carbon atoms,
SO₃H group, optionally protected by a pentyl group,
PO₃H₂ group, optionally substituted by a 5 (-CH₂CH₂SCOR₅) group, with R₅ representing a C₁-C₄ alkyl group, a phenyl or benzyl group, or
tetrazolyl group.
R₂ denotes an alkyl chain, or an aryl, arylalkyl, cycloalkyl, (cycloalkyl)alkyl, (heteroaryl)alkyl group which may or may not be substituted by at least one OH, OR, SR′, NH₂, NHR′, guanidinyl, COOH or CONH₂ group, or a halogen atom selected from among F, Cl, Br or I with R′ representing a straight-chain or branched C₁₋₄ alkyl group.
R₃ denotes a hydrogen atom or a methyl group,
R₄ denotes
an alkyl chain, an aryl, arylalkyl, cycloalkyl, (cycloalkyl)alkyl, (heteroaryl)alkyl, heterocycloalkyl or (heterocycloalkyl)alkyl group substituted by at least one CONH₂, SO₃H, SO₂NH₂, PO₃H₂ or tetrazolyl group, with the groups SO₃H and PO₃H₂ optionally protected,
a C₂₋₆ alkyl chain, an aryl, arylalkyl, cycloalkyl, (cycloalkyl)alkyl, (heteroaryl)alkyl, heterocycloalkyl or (heterocycloalkyl)alkyl group substituted by at least one CO₂H group optionally protected, or
R₃ and R₄ may together constitute a 5- or 6-membered heterocyclic compound, substituted by at least one CO₂H, CONH₂, SO₃H, SO₂NH₂ or PO₃H₂ group with the groups CO₂H, SO₃H and PO₃H₂ optionally protected,
X denotes a group CONH or CH₂NH,
Z denotes a group OH, OCH₂-C₆H₅ or NR″R′″ wherein R″ and R′″ independently of one another may denote a hydrogen atom or an alkyl, aryl or arylalkyl group, where R″ and R′″ may constitute, together with the nitrogen atom, a 5- or 6-membered heterocycle possibly having a second heteroatom selected from among O, S and N, and
R denotes a hydrogen atom or a group of formula II 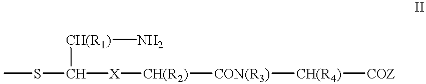
corresponding to the symmetric disulphide of the inhibitor wherein R₁, R₂, R₃, R₄, X and Z are as hereinbefore defined,
and the derivatives thereof.

16. A method for the prevention or treatment of anxiety states or panic attacks, essential and secondary arterial hypertension, cardiac and renal failure, disorders of hydrodynamic homeostasis, myocardial infarct and proteinuria in diabetics, comprising administering to a patient in need of such treatment a therapeutically efficient amount of a compound of general formula (I): 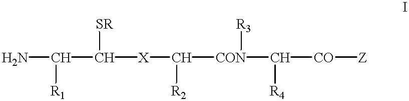
wherein
R₁ denotes an alkyl, alkenyl or alkynyl chain, or a cycloalkyl or (cycloalkyl)alkyl group substituted by at least one
—COOH group, optionally esterified by an alkyl group comprising 2 to 12 carbon atoms,
SO₃H group, optionally protected by a pentyl group,
PO₃H₂ group, optionally substituted by a (—CH₂CH₂SCOR₅) group, with R₅ representing a C₁-C₄ alkyl group, a phenyl or benzyl group, or
tetrazolyl group.
R₂ denotes an alkyl chain, or an aryl, arylalkyl, cycloalkyl, (cycloalkyl)alkyl, (heteroaryl)alkyl group which may or may not be substituted by at least one OH, OR, SR′, NH₂, NHR′, guanidinyl, COOH or CONH₂ group, or a halogen atom selected from among F, Cl, Br or I with R′ representing a straight-chain or branched C₁₋₄ alkyl group.
R₃ denotes a hydrogen atom or a methyl group,
R₄ denotes
an alkyl chain, an aryl, arylalkyl, cycloalkyl, (cycloalkyl)alkyl, (heteroaryl)alkyl, heterocycloalkyl or (heterocycloalkyl)alkyl group substituted by at least one CONH₂, SO₃H, SO₂NH₂, PO₃H₂ or tetrazolyl group, with the groups SO₃H and PO₃H₂ optionally protected,
a C₂₋₆ alkyl chain, an aryl, arylalkyl, cycloalkyl, (cycloalkyl)alkyl, (heteroaryl)alkyl, heterocycloalkyl or (heterocycloalkyl)alkyl group substituted by at least one CO₂H group optionally protected, or
R₃ and R₄ may together constitute a 5- or 6-membered heterocyclic compound, substituted by at least one CO₂H, CONH₂, SO₃H, SO₂NH₂ or PO₃H₂ group with the groups CO₂H, SO₃H and PO₃H₂ optionally protected,
X denotes a group CONH or CH₂NH,
Z denotes a group OH, OCH₂-C₆H₅ or NR″R′″ wherein R″ and R′″ independently of one another may denote a hydrogen atom or an alkyl, aryl or arylalkyl group, where R″ and R′″ may constitute, together with the nitrogen atom, a 5- or 6-membered heterocycle possibly having a second heteroatom selected from among O, S and N, and
R denotes a hydrogen atom or a group of formula II 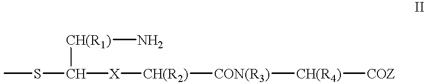
corresponding to the symmetric disuilphide of the inhibitor wherein R₁, R₂, R₃, R₄, X and Z are as hereinbefore defined,
and the derivatives thereof.

17. A method of diagnosis of anxiety states or panic attacks, essential and secondary arterial hypertension, cardiac and renal failure, disorders of hydrodynamic homeostasis, myocardial infarct and proteinuria in diabetics, wherein the aminopeptidase A is evaluated in a biological sample of a patient to be tested by using a compound of formula (I): 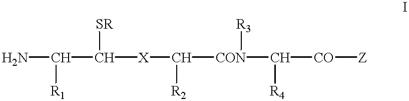
wherein:
R₁ denotes an alkyl, alkenyl or alkynyl chain, or a cycloalkyl or (cycloalkyl)alkyl group substituted by at least one
—COOH group, optionally esterified by an alkyl group comprising 2 to 12 carbon atoms,
SO₃H group, optionally protected by a pentyl group,
PO₃H₂ group, optionally substituted by a (—CH₂CH₂SCOR₅) group, with R₅ representing a C₁-C₄ alkyl group, a phenyl or benzyl group, or
tetrazolyl group.
R₂ denotes an alkyl chain, or an aryl, arylalkyl, cycloalkyl, (cycloalkyl)alkyl, (heteroaryl)alkyl group which may or may not be substituted by at least one OH, OR, SR′, NH₂, NHR′, guanidinyl, COOH or CONH₂ group, or a halogen atom selected from among F, Cl, Br or I with R′ representing a straight-chain or branched C₁₋₄ alkyl group.
R₃ denotes a hydrogen atom or a methyl group,
R₄ denotes
an alkyl chain, an aryl, arylalkyl, cycloalkyl, (cycloalkyl)alkyl, (heteroaryl)alkyl, heterocycloalkyl or (heterocycloalkyl)alkyl group substituted by at least one CONH₂, SO₃H, SO₂NH₂, PO₃H₂ or tetrazolyl group, with the groups SO₃H and PO₃H₂ optionally protected,
a C₂₋₆ alkyl chain, an aryl, arylalkyl, cycloalkyl, (cycloalkyl)alkyl, (heteroaryl)alkyl, heterocycloalkyl or (heterocycloalkyl)alkyl group substituted by at least one CO₂H group optionally protected, or
R₃ and R₄ may together constitute a 5- or 6-membered heterocyclic compound, substituted by at least one CO₂H, CONH₂, SO₃H, SO₂NH₂ or PO₃H₂ group with the groups CO₂H, SO₃H and PO₃H₂ optionally protected,
X denotes a group CONH or CH₂NH,
Z denotes a group OH, OCH₂-C₆H₅ or NR″R′″ wherein R″ and R′″ independently of one another may denote a hydrogen atom or an alkyl, aryl or arylalkyl group, where R″ and R′″ may constitute, together with the nitrogen atom, a 5- or 6-membered heterocycle possibly having a second heteroatom selected from among O, S and N, and
R denotes a hydrogen atom or a group of formula II 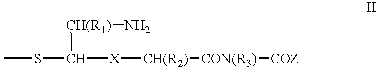
corresponding to the symmetric disulphide of the inhibitor wherein R₁, R₂, R₃, R₄, X and Z are as hereinbefore defined,
and the derivatives thereof,
and is compared to the level present in normal subjects.
